(19) United States
(12) Plant Patent Application Publication
(10) Pub. No.: US 2003/0110542 P1
(43) Pub. Date: Jun. 12, 2003

(54) Miniature rose plant named 'Savamont'

(76) Inventor: John M. Saville, Newburyport, MA (US)

Correspondence Address: CHRISTIE, PARKER & HALE, LLP, 350 WEST COLORADO BOULEVARD, SUITE 500, PASADENA, CA, 91105, US

(21) Appl. No.: 10/015,143

(22) Filed: Dec. 10, 2001

(51) Int. Cl. A01H 5/00

(52) U.S. Cl. PLT/122

(57) ABSTRACT

A new and distinct Miniature Rose plant having attractive Red flowers.

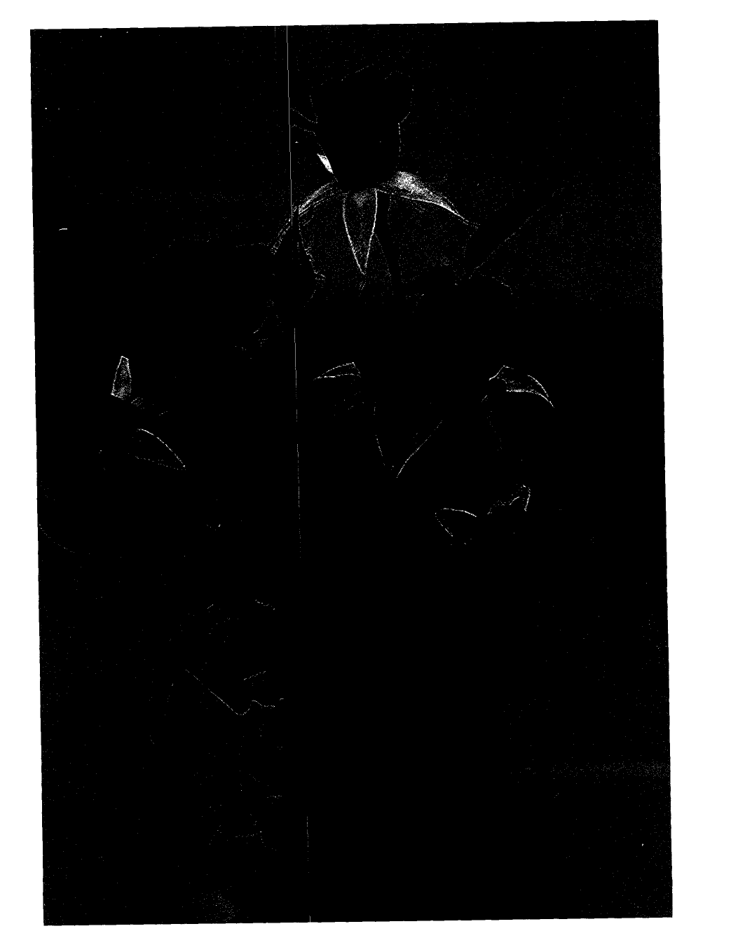

### BACKGROUND OF THE INVENTION

The present invention relates to a new and distinct variety of miniature Rose class. The new variety was created by crossing as seed parent the variety known as ‘BENALAV’ (U.S. Plant Pat. No. 6,517) and as pollen parent, the variety known as “RUMIRED’ (U.S. Plant Pat. No. 5,770). The varietal denomination of the new variety is ‘SAVAMONT’.

### SUMMARY OF THE INVENTION

Among the novel characteristics possessed by the new variety which distinguishes it from its parents and all other varieties of which I am aware, is its attractive Red flowers.

Asexual reproduction by propagation of vegetative cuttings of the new variety as performed in Essex County, Massachusetts show that the foregoing and other distinguishing characteristics come true to form and are established and transmitted through succeeding propagation.

### COMPARISON WITH PARENTS

The new variety may be distinguished from its parents in flower color. The new variety is much taller and more upright than its pollen parent ‘RUIMIRED’ and whereas the flower of the new variety is more Blue Red in color, flower of ‘RUIMIRED’ is more of an Orange Red color.

### BRIEF DESCRIPTION OF ILLUSTRATION

The accompanying photograph illustrates a typical specimen of the new variety and shows the flowering thereof from bud to full bloom depicted in color as nearly true as it is reasonably possible to make in a color illustration of the character. Throughout this specification, color references and/or values are based upon The Royal Horticultural Society Colour Chart and terminology used in the color descriptions herein refers to plate numbers in this color chart except where common terms of color definition are employed.

### DESCRIPTION OF THE NEW VARIETY

The following observations were made of 5 year-old plants of the new variety grown in 2-gallon plastic containers in Essex County, Massachusetts during the months of July and August. Phenotypic expression may vary with environmental, cultural and climatic conditions, as well as differences in conditions of light and soil.

### FLOWER

Blooming Habit: Continuous; flowers remain attached dropping with petals and peduncle after fade

Bud.—Medium; length about ½ to ⅝ inch just before sepals divide Form: Obovate Color: When sepals first divide color near 60A; when petals begin to unfurl, color near 60A.

Sepals.—Lanceolate with occasional flags; extending near ⅝ inch beyond tip of bud Color: Upper Surface: Near 144A Under surface: Near 147C Hip/Stem Position: Rolling back quickly to 45° angle with stem

Peduncle.—Length: About 1 to 1½ inches Aspect: Straight Strength: Erect. Color: Near 144A; texture smooth.

### BLOOM

Size: Average when fully expanded 1⅞ inches

Borne: Clusters of 2 to 8 and also singly

Form: Opens flat becoming and remaining loosely rolled outward

Petalage: Between 24 to 30

Fragrance: Slight

Persistence: Drop off cleanly

Lasting Quality: Between 7 to 10 days as cut flower

### PETALS

Texture: Smooth

Appearance: Inside velvety; outside satiny with slightly raised veins

Form: Broad, spatulate

Size: Length about 11/16 inch at point of attachment to tip; outer petal width about ¾ inch

Arrangement: Shingle-like; regularly arranged

Color:

During first 2 days.—Petals at center of flower upper side near 46A, under side near 53A; outer petals upper side near 46A, under side near 60A.

In full bloom.—Inner petals upper side between 60A and 53A, underside near 53A; outer petals upper side near 53A, under side near 60A.

Base of petals (Point of attachment): White

General tonality from a distance: Dark Red

Color Change: Very slight as bloom ages

General tonality from a distance.—Dark Red even after 3 or more days.

Petaloids in center: About 2 to 6

Color.—Same as inner petals.

Texture.—Smooth.

### REPRODUCTIVE ORGANS

Arrangement of stamens:

Filaments and Anthers: Regularly arranged around styles

Stamens: About 100 or more

Filaments: About 1/16 to ⅛ inch; color Deep Yellow near 20A

Anthers: Near 20B

Pollen: Ample quantity; color near 17A

Pistils: About 30

Styles: Thin, uneven length between 1/16 to 3/16 inch; color near 53C

Stigmas: Near 27B

Receptacle:

Shape.—Round; surface texture pubescent.

Size.—About 5/16 inch.

Color.—Off-White near 196D.

Hips:

Shape.—Mostly globular, occasionally apple and pear shaped.

Size.—Average diameter at widest point between 13/16 and 14/16 inch.

Color.—Some areas near 28B; some areas near 167A.

Seeds: Seeds do not protrude; surface texture glabrous; color near 19C.

### PLANT

Form: Upright, compact

Growth: Vigorous, uniform branching

Size: At maturity, height between 18 to 21 inches; width between 20 to 24 inches

Foliage: Generally 5 leaflets, occasionally 3 or 7 leaflets

Size.—About 3½ inches from stem to tip measured along the rachis; about 1¾ inches on mature leaflet at terminal leaflet base to tip.

Quantity.—Abundant.

Color.—New foliage: Upper side near 146A flushed with 187A; under side near 146B flushed with 187A Mature foliage: Upper side near 147A; under side near 147B

Shape.—Oval pointed.

Texture.—Upper side: Semi-glossy and glabrous Under side: Matte and glabrous.

Edge.—Serrated.

Serration.—Fine on new foliage becoming somewhat coarse and slightly uneven on older foliage.

Stipules: Very finely bearded; length is about ½ the length to the first set of leaflets; upper surface color near 147B, under surface near 146C

Rachis: Smooth texture; upper side color near 147A, under side near 144A

Prickles.—Occasional; between 2 to 3 prickles on mature leaves, occasionally 5; color near 144C.

Resistance to disease: Good resistence to insects and Mildew; average resistance to Blackspot, resistance to Rust not observed

### WOOD

New shoots:

Bark.—Smooth.

Wood.—Near 144A.

Old wood:

Bark.—Smooth.

Wood.—Near 146A.

Flowering stem: Length between 4½ to 5½ inches

Thorns:

Quantity.—Ordinary quantity on main stalks from base; many thorns on laterals from stalk; fewer closest to bud/flower.

Form.—Flat base, slightly curved downward to a point; irregularly positioned.

Length.—About ¼ inch.

Color.—Immature thorns: Tip near 161C, at the base near 183D. Mature thorns: Near 165B.

Prickles.—None on main stalks.

Winter Hardiness: Tested hardy zones 4 to 9; winter protection recommended in Zones 5 and colder

I claim:

1. A new and distinct Miniature Rose plant of the variety substantially as shown and described.
